(19) United States
(12) Patent Application Publication
(10) Pub. No.: US 2002/0022592 A1
(43) Pub. Date: Feb. 21, 2002

(54) Delivery of thrombospondin from implantable tissue matrices

(76) Inventors: Michael Detmar, Arlington, MA (US); Joseph P. Vacanti, Winchester, MA (US); Michael Streit, Boston, MA (US); Antonia E. Stephen, Boston, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(21) Appl. No.: 09/822,161

(22) Filed: Mar. 30, 2001

Related U.S. Application Data
(63) Continuation-in-part of application No. 09/536,087, filed on Mar. 24, 2000.
(63) Continuation-in-part of application No. 09/770,339, filed on Jan. 26, 2001.
(60) Provisional application No. 60/127,221, filed on Mar. 31, 1999.
(60) Provisional application No. 60/178,842, filed on Jan. 27, 2000.

(51) Int. Cl. A61K 45/00; A61K 38/48

(52) U.S. Cl. 514/12; 424/93.7

(57) ABSTRACT

Normal cells, such as fibroblasts or other tissue or organ cell types, are genetically engineered to express biologically active, anti-angiogenic compounds, in particular, thrombospondin-2. These cells are seeded into a matrix for implantation into the patient to be treated. Cells may also be engineered to include a lethal gene, so that implanted cells can be destroyed once treatment is completed. Cells can be implanted in a variety of different matrices. In a preferred embodiment, these matrices are implantable and biodegradable over a period of time equal to or less than the expected period of treatment, during which the engrafted cells form a functional tissue producing the desired biologically active agent for longer periods of time. These devices and strategies are used as delivery systems, which may be implanted by standard or minimally invasive implantation techniques, for delivery of anti-angiogenic molecules, especially thrombospondin-2, for the treatment of a variety of conditions that produce abnormal growth, including treatment of malignant and benign neoplasias, vascular malformations (hemangiomas), inflammatory conditions, keloid formation and adhesion, endometriosis, congenital or endocrine abnormalities, and other conditions that can produce abnormal growth such as infection.

This application claims priority to U.S. Ser. No. 09/536,087 filed Mar. 24, 2000, which claims priority to U.S. Ser. No. 60/127,221, filed Mar. 31, 1999, and to U.S. Ser. No. 09/770,339 filed Jan. 26, 2001, which claims priority to U.S. Ser. No. 60/178,842 filed Jan. 27, 2000.

The United States government has rights in this invention by virtue of NIH/NCI grants CA 69184 and CA 86410 to Michael Detmar, by Department of Defense grant 1200-202487 to Joseph P. Vacanti.

### BACKGROUND OF THE INVENTION

The present invention is generally in the area of methods and systems for treatment of disorders such as cancer with angiogenesis inhibitors, using genetically engineered host cells or natural cells to secrete the angiogenesis inibitors following implantation into the patient using biodegradable polymeric matrices.

One of the difficulties in treatment of conditions such as cancer using protein or other biological modifiers is the need for large quantities of the therapeutic agent to be delivered over an extended period of time. For most of the compounds discovered during research on complex pathways or unique tissues, it has not been possible, or has not been commercially feasible, to produce the compounds in sufficient quantity to treat the disorders. Numerous examples of these compounds, especially proteins, have been reported.

Endogenous anti-angiogenic factors are generally thought to maintain the quiescence of the mature vasculature by counterbalancing the activity of angiogenesis inducers, Carmeliet, D., et. al., Nature 407, 249-257 (2000). Malignant tumors have to overcome the activity of angiostatic factors in order to induce and to sustain angiogenesis which is essential for tumor growth, invasion and metastasis. Several endogenous anti-angiogenic factors, including angiostatin (O'Reilley, M., et al., Nature Med. 2, 689-692 (1996)), endostatin (O'Reilly, M. S. et al. Cell 88, 277-285 (1997)) and vasostatin (Pike, S. E. et al. J. Exp. Med. 188, 2349-2356 (1998)), have been shown to inhibit tumor angiogenesis, tumor growth, and metastasis in experimental tumor models. Because of difficulties with the stability and high costs associated with the large-scale production of recombinant forms of endogenous angiogenesis inhibitors, anti-angiogenic gene therapy has become a rapidly expanding field (Feldman, A. L., et al., Cancer 89, 1181-1194 (2000)). Moreover, experimental tumor studies suggest that the pharmacokinetics after bolus administration of recombinant angiostatic factors are not optimal for maintaining a sustained anti-angiogenic effect on tumor vessels, as compared with continuous delivery of anti-angiogenic agents (Drixler, T. A. et al., Cancer Res. 60, 1761-1765 (2000), Crystal, R. G. Nat. Biotechnol. 17, 336-337 (1999), and Hahnfeldt, P., et al.,. Cancer Res. 59, 4770-4775 (1999)).

The matricellular protein thrombospondin-2 (TSP-2) was recently identified as a potent endogenous inhibitor of tumor growth and angiogenesis Streit, M. et al. Proc Natl. Acad. Sci. (USA) 96, 14888-14893 (1999). When overexpressed TSP-2 in human cutaneous squamous cell carcinomas, it was found to be a potent inhibition of orthotopic tumor growth and to significantly reduce tumor vascularization. TSP-2 is a 420 kD matricellular protein that inhibits the angiogenic activity of fibroblast growth factor, Volpert, O. V. et al., Biochem. Biophys. Res. Commun. 217, 326-332 (1995), and the formation of focal adhesions in bovine endothelial cells, Murphy-Ullrich, J. E., et al., J. Biol. Chem. 268, 26784-26789 (1993), in vitro. TSP-2 is a member of a multigene family of five secreted, modular glycoproteins and has a considerable structural similarity to the endogenous angiogenesis inhibitor thrombospondin-1 (TSP-1) Lawler, J. Curr. Opin. Cell Biol. 12, 634-640 (2000). However, recent results demonstrated that induced TSP-2 expression resulted in a significantly stronger anti-angiogenic and anti-tumoral effect than induced TSP-1 expression Streit, M, et al. Proc. Natl. Acad. Sci. USA 96(26):14888-14893 (1999).

Tumors need to induce neovascularization to support malignant growth and metastasis. Anti-angiogenic therapy selectively targets microvascular endothelial cells recruited into the tumor bed, thereby avoiding some of the adverse effects and the induction of acquired drug resistance commonly observed with conventional chemotherapy, Kerbel, R. S. Carcinogenesis 21, 505-515 (2000). The efficiency of anti-angiogenic tumor therapy has been confirmed in a large number of in vivo tumor studies. However, a prolonged protein therapy with angiogenesis inhibitors such as angiostatin or endostatin in repeated cycles is associated with high costs, and recent evidence suggest that the continuous delivery of endogenous angiogenesis inhibitors is more efficient than single dosing. This may be achieved most effectively by anti-angiogenic gene therapy.

Viral and non-viral experimental gene therapy systems have been developed to achieve prolonged circulating serum levels of endogenous anti-angiogenic factors. However, repeated cycles of therapy are required to maintain elevated anti-angiogenic protein levels. Moreover, adenoviral therapy might elicit strong host immune responses after repeated injections, thereby significantly reducing its long-term therapeutic efficiency Anderson, W. F., Human gene therapy. Nature 392 (6679 Suppl.), 25-30 (1998).

It is therefore an object of the present invention to provide methods and reagents for production of clinically effective amounts of therapeutic biologicals, especially anti-angiogenic proteins such as thrombospondin-2, in vivo.

It is a still further object of the present invention to provide methods and reagents for treatment of a variety of disorders characterized by the proliferation of abnormal tissue, including malignant and benign neoplasias, vascular malformations, inflammatory conditions including restenosis, infection, keloid formation and adhesions, congenital or endocrine abnormalities, and other conditions that produce abnormal growth, using anti-angiogenic inhibitors expressed by genetically engineered cells implanted on and within a matrix into a patient in need thereof.

### SUMMARY OF THE DISCLOSURE

Normal cells, such as fibroblasts or other tissue or organ cell types, are genetically engineered to express biologically active, anti-angiogenic compounds, in particular, thrombospondin-2. These cells are seeded into a matrix for implantation into the patient to be treated. Cells may also be engineered to include a lethal gene, so that implanted cells can be destroyed once treatment is completed. Cells can be implanted in a variety of different matrices. In a preferred embodiment, these matrices are implantable and biodegradable over a period of time equal to or less than the expected period of treatment, during which the engrafted cells form a functional tissue producing the desired biologically active agent for longer periods of time. Representative cell types include fibroblasts, tissue specific cells, progenitor cells, and stem cells. Matrices can be formed of synthetic or natural materials, by chemical coupling at the time of implantation, using standard techniques for formation of fibrous matrices from polymeric fibers, and using micromachining or microfabrication techniques.

The matrices are used as delivery systems, which may be implanted by standard or minimally invasive implantation techniques, for the treatment of a variety of conditions that produce abnormal growth, including treatment of malignant and benign neoplasias, vascular malformations (hemangiomas), inflammatory conditions, keloid formation and adhesion, endometriosis, congenital or endocrine abnormalities, and other conditions that can produce abnormal growth such as infection. Efficacy of treatment with the anti-angiogenic compound is detected by determining specific criteria, for example, cessation of cell proliferation, regression of abnormal tissue, or cell death.

Recent studies indicate that continuous delivery of endogenous angiogenesis inhibitors for cancer treatment is more potent than intermittent dosing, suggesting a potential role of gene therapy in anti-angiogenic tumor therapy. As demonstrated by the examples, a tissue-engineered implant system for the continuous in vivo production of thrombospondin-2 (TSP-2), a potent endogenous inhibitor of tumor growth and angiogenesis, provides a means for effective, continuous dosing in vivo. Fibroblasts were retrovirally transduced to overexpress TSP-2 and were seeded onto biodegradable polymer scaffolds. After transplantation into the peritoneal cavity of nude mice, bioimplants maintained high levels of TSP-2 secretion over extended time periods, resulting in increased levels of circulating TSP-2. Bioimplant-generated TSP-2 potently inhibited tumor growth and angiogenesis of human squamous cell carcinomas, malignant melanomas and Lewis lung carcinomas that were implanted at a distant site. These results confirm the therapeutic efficiency of cell-based anti-angiogenic gene therapy using tissue engineered bioimplants.

### BRIEF DESCRIPTION OF THE DRAWINGS

FIG. 1 is a schematic of the engineering of cells with TSP-2 retroviral vectors, which are seeded onto a biodegradable mesh, cultured fourteen days, then implanted into mice injected with A431, B16F10, MDA MB435-GFP, or Lewis Lung carcinoma cells.

FIGS. 2A, 2B and 2C are figures showing tumor volume (mm³) over time in days, comparing tumor volumes following exposure to control cells versus cells genetically engineered to express TSP-2, for A431 squamous cell carcinomas (FIG. 2A), B16F10 malignant melanomas (FIG. 2B), and Lewis Lung Carcinomas (FIG. 2C). The graphs show that circulating TSP-2 significantly (P<0.001) inhibited the tumor growth of A431 squamous cell carcinomas (FIG. 2A), B16F10 malignant melanomas (FIG. 2B) and LLCs (FIG. 2C). Tumor cells were injected 1 week after the implantation of TSP-2 secreting fibroblast-grafts (TSP-2) or control fibroblast-grafts (control). Values represent means for two separate tumors per mouse and time point.

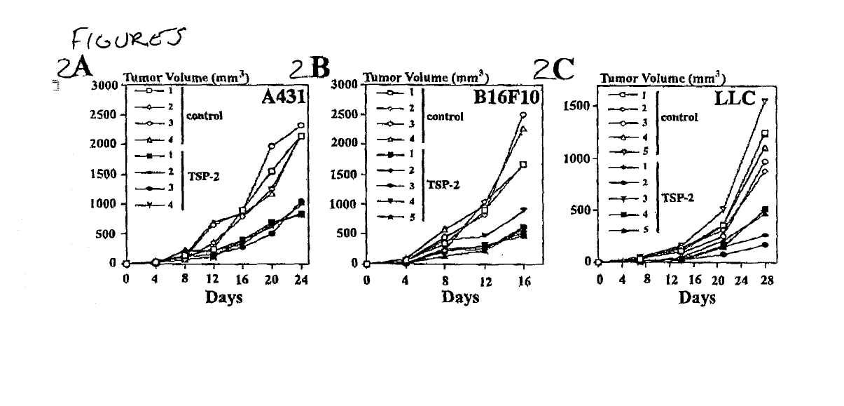

FIGS. 3A, 3B and 3C are graphs of number of vessels/mm² (FIG. 3A), average vessel size (microns²), and total vascular area (%), for A431, B16F10, and LLC. Quantitative computer-assisted image analysis of tumor-associated blood vessels revealed a marked inhibition of tumor angiogenesis by systemic TSP-2, as shown by the reduced average density of blood vessels per mm² tumor area (FIG. 3A), the significant (P<0.01) reduction of the average blood vessel size (FIG. 3B) and the decreased (P<0.01) relative tumor area covered by blood vessels (FIG. 3C). CD31-stained blood vessels were evaluated in three different ×10 fields in sections obtained from five different tumors of each tumor cell line in mice bearing TSP-2 overexpressing (TSP-2) or control fibroblast grafts (control).

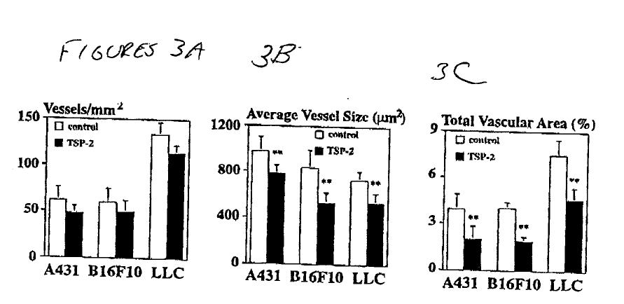

### DETAILED DESCRIPTION OF THE INVENTION

A strength of biological modifiers is that they impart specificity to treatment paradigms to allow for prolonged parenteral therapy, and eliminate many of the side effects and inconveniences associated with conventional therapies. Problems which are often encountered with these molecules include purification and enrichment. Most are manufactured in the laboratory using recombinant technology. A small number are selected for scale up by the pharmaceutical industry. As an important step for their purification, they undergo rigorous purification schemes using separation methods that may alter their chemical characteristics. As is often the case, the end product is a small percentage of the starting material, and is frequently less potent. To obviate the loss of quantity and potency, a polymer scaffolding or matrix has been used to proliferate cells producing the biological modifiers. When this scaffold or matrix is implanted into an organism, it becomes vascularized or otherwise connected to the vasculature, the seeded cells grow to fill the scaffold, the biological modifiers are secreted directly into the bloodstream or adjacent cells, and, in a preferred embodiment, the scaffold is resorbed, leaving a new secretory tissue. Elimination of the purification steps enhances yield and avoids the problems with contamination, cost and loss of biological activity.

I. Materials for Production of Secretory Tissues

The materials required for production in vivo of biologically active molecules include cells which produce the biologically active molecules and matrices for proliferation of the engineered cells which can be implanted in vivo to form new secretory tissues. In one embodiment, cells are obtained which already produce the desired biological modifiers. In another embodiment, cells are genetically engineered to produce the biological modifiers. In this embodiment, it is also necessary to provide the appropriate genes, means for transfection of the cells, and means for expression of the genes.

A. Cells for Production of Cell Implants

Cells to be engineered can be obtained from established cell culture lines, by biopsy or from the patient or other individuals of compatible tissue types. The preferred cells are those obtained from the patient to be treated. In those cases where the patient's own cells are not used, the patient will also be treated with appropriate immunosuppressants such as cyclosporine to avoid destruction of the implanted cells during therapy.

In the preferred embodiments, cells are obtained directly from the donor, washed, and cultured using techniques known to those skilled in the art of tissue culture. Cells are then transfected with the gene of interest and seeded at various cell counts onto a matrix such as a polymeric mesh to achieve optimal production of the anti-angiogenic compound.

Cell attachment and viability can be assessed using scanning electron microscopy, histology, and quantitative assessment, for example, by ELISA, fluorescent labelled or radioactive labelled antibodies. The function of the implanted cells can be determined using a combination of the above-techniques and functional assays. Studies using protein assays can be performed to quantitate cell mass on the polymer scaffolds. These studies of cell mass can then be correlated with cell functional studies to determine the appropriate cell mass.

B. Biologically Active Anti-angiogenic Molecules

Any biologically active anti-angiogenic molecule which has been cloned or for which a cellular source is available can be used. The preferred compound is thrombospondin. In order to grow beyond minimal size and to metastasize, tumors need to induce the growth of new blood vessels (angiogenesis) providing a lifeline for tumor sustenance and waste disposal (Hanahan and Folkman J Cell 1996, 86:353-64). Tumor development is associated with increased release of angiogenesis factors, most prominently of vascular endothelial growth factor (VEGF) (Brown LF et al., Exs 1997, 79:233-69). Several studies have shown that overexpression of angiogenesis factors in experimental tumors leads to enhanced tumor growth and vascularization, and therapeutic inhibition of VEGF activity has been shown to inhibit tumor growth and metastasis (Ferrara et al., Breast Cancer Res Treat 1995, 36:127-37; Claffey et al. Cancer Res 1996, 56:172-18; Skobe et al., Nat Med 1997, 3:1222-7).

Several naturally occurring angiogenesis inhibitors have been identified, including thrombospondin-1 (TSP-1) (Iruela-Arispe et al., Proc Natl Acad Sci USA 1991, 88:5026-5030), angiostatin (O'Reilly et al., Cell 1994, 79:315-28) and endostatin (O'Reilly et al., Cell 1997, 88:277-85). TSP-1 is a 420 kd homotrimeric matricellular glycoprotein that regulates attachment, proliferation, migration and differentiation of various cell types (Bornstein et al., Cell Biol 1995, 130:503-506). TSP-1 inhibits proliferation and migration of vascular endothelial cells in vitro and inhibits neovascularization in vivo, contributing to the normal quiescence of the vasculature (Tolsma et al., Cell Biol 1993, 122:497-511). TSP-1 protein expression was shown to be inversely correlated to cellular differentiation in several squamous cell carcinoma (SCC) cell lines (Goodson et al, Proc Natl Acad Sci USA 1994, 91:7129-7133), and was shown to induce SCC proliferation, adhesion, migration and invasion of cells in vitro (Siemeister et al., Cancer Metastasis Rev 1998, 17:241-248; Bornstein FASEB J 1992, 6:3290-3299; Gorczyca et al. Cancer Res 1993, 53:1945-51). Enhancement of in vitro tumor cell invasion by TSP-1 has also been reported for breast, lung and pancreatic carcinoma cell lines (Albo et al. Biochem Biophys Res Commun 1994, 203:857-65; Robbins et al.: Arch Pathol Lab Med 1987, 111:841-5; Albo et al.: Surgery 1997, 122:493-500; Christofori Angiogenesis 1998, 2:21-23).

The goal is not to form a permanent new tissue, but to provide an implanted “bioreactor” to produce therapeutic biologicals for a defined period effective to cause cessation of cell proliferation, regression of abnormal tissue, or cell death.

In the preferred embodiment, the cells are engineered to express an isolated nucleic acid molecule which comprises the coding region of TSP-2, or a sequence which encodes a fragment or a peptide-based analog of TSP-2. The nucleic acid can include a 5′ or 3′ nucleic acid sequence not present in the native TSP-2 human sequence. In one embodiment, the nucleic acid encoding human TSP-2 includes a functional regulatory sequence, e.g., a 5′ and/or a 3′ sequence which modulates expression of TSP-2. In one embodiment the control sequence can be an endogenous regulatory sequence. In another embodiment the regulatory sequence can be a heterologous regulatory sequence. The heterologous regulatory sequence can be a human or non-human regulatory sequence, or a combination of both. A regulatory sequence can include one or more elements of a regulatory sequence, e.g., the regulatory sequence can include a promoter, an enhancer, an insulator, or a DNA binding element. Suitable peptides and nucleic acid sequences are described in U.S. Ser. No. 09/09/536,087 filed Mar. 24, 2000, the teachings of which are incorporated herein.

Although described primarily as to engineering of cells to express the anti-angiogenic molecule, such as TSP-2, it is also possible to increase expression of the endogenous anti-angiogenic molecule. For example, the level of TSP-2 can be increased by increasing the endogenous TSP-2 activity by increasing the level of expression of the gene, e.g., by increasing transcription of the TSP-2 gene; increasing the stability of the TSP-2 mRNA, e.g., by altering the secondary or tertiary structure of the mRNA; increasing the translation of TSP-2 mRNA, e.g., by altering the sequence of the TSP-2 mRNA; and/or increasing the stability of the TSP-2 protein. Transcription of the TSP-2 gene can be increased, e.g., by altering the regulatory sequences of the endogenous TSP-2 gene. In one embodiment the regulatory sequence can be altered by: the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the TSP-2 gene to be transcribed more efficiently.

C. Vectors for Engineering Cells

Examples of recombinant DNA techniques include cloning, mutagenesis, and transformation. Recombinant DNA techniques are disclosed in Maniatis et al., Molecular Cloning: A Laboratory Manual, Cold Spring Harbor, N.Y. (1982). Vectors, including adeno-associated viruses, adenoviruses, retroviruses, and tissue specific vectors, are commercially available. Vectors can include secretory sequences, so that the biological modifier will diffuse out of the cell in which it is expressed and into the vascular supply or interstitial spaces in order to expose the cells of interest to concentrations of the protein that are effective to treat the patient. The vector or expression vehicle, and in particular the sites chosen therein for insertion of the selected DNA fragment and the expression control sequence, are determined by a variety of factors, e.g., number of sites susceptible to cleavage by a particular restriction enzyme, size of the protein to be expressed, expression characteristics such as start and stop codons relative to the vector sequences, and other factors recognized by those of skill in the art. The choice of a vector, expression control sequence, and insertion site for DNA sequence encoding the biological modifier is determined by a balance of these factors.

It should be understood that the DNA sequences coding for the biological modifier that are inserted at the selected site of a cloning or expression vehicle may include nucleotides which are not part of the actual gene coding for the biological modifier or may include only a fragment of the actual gene. It is only required that whatever DNA sequence is employed, a transformed host cell will produce the biological modifier. For example, DNA sequences may be fused in the same reading frame in an expression vector with at least a portion of a DNA sequence coding for at least one eukaryotic or prokaryotic signal sequence, or combinations thereof.

The vector can be a plasmid vector or a viral vector. The vector can be circular or linear. In a preferred embodiment, the vector can include one or more of the following elements, e.g., an origin of replication, a promoter, and a selection marker, e.g., a drug resistance marker. A viral vector can be a retrovirus, an adenovirus, an adeno-associated virus, an SV40 virus, or a herpes virus. Retroviral vectors are particularly useful, as they selectively integrate into the genome of replicating cells, such as tumour cells. Alternatively non-viral vectors can be used, e.g., pCDM8 (Seed (1987) Nature 329:840) and pMT2Pc (Kaufman. (1987) EMBO J. 6:187-195).

The vector can be introduced into a host cell by standard transfection techniques, e.g., electroporation, microinjection, calcium phosphate precipitation, modified calcium phosphate precipitation, polybrene precipitation, liposome fusion, receptor-mediated DNA delivery). The vector can remain episomal, or can be incorporated into the genome of the host cell.

D. Matrices

There are three basic types of matrices that can be used: devices formed by micromachining, micromolding or other microfabrication techniques, fibrous polymeric scaffolds, and hydrogels.

1. Microfabricated Device Design and Manufacture

Preferred materials for making devices to be seeded with cells are biodegradable polymers, although in some embodiments non-degradable materials may be preferred or may be used as structural support or as components of a device formed of biodegradable polymer. The polymer composition can be selected both to determine the rate of degradation as well as to optimize proliferation. Many biodegradable, biocompatible polymeric materials can be used to form the device, or guide channels within the device, including both natural and synthetic polymers, and combinations thereof. Examples of natural polymers include proteins such as collagen, collagen-glycosaminoglycan copolymers, polysaccharides such as the celluloses (including derivatized celluloses such as methylcelluloses), extracellular basement membrane matrices such as Biomatrix, and polyhydroxyalkanoates such as polyhydroxybutyrate (PHB) and polyhydroxybutyrate-co-valerate (PHBV) which are produced by bacterial fermentation processes. Synthetic polymers include polyesters such as polyhydroxyacids like polylactic acid (PLA), polyglycolic acid (PGA) and compolymers thereof (PLGA), some polyamides and poly(meth)acrylates, and polyanhdyrides. Examples of non-degradable polymers include ethylenevinylacetate (EVA), polycarbonates, and some polyamides.

The surface morphology of the devices can affect cell growth. Bioactive materials may also be incorporated into the device or a sustained release matrix within the device to promote cell viability or proliferation. These materials can be incorporated into the polymer at a loading designed to release by diffusion and/or degradation of the polymer forming the device over a desired time period, ranging from days to weeks. Alternatively, the bioactive substance may be incorporated into a matrix loaded into or adjacent to the device. These matrices may be formed of the same materials as the device or may consist of polymeric materials incorporated within the tracts or channels, for example, hydrogel matrices of the types described in the literature (for example, Wells, et al., Exp. Neurol. (1997) 146(2):395-402; Chamberlain, et al., Biomaterials 1998 19(15):1393-1403; and Woerly, et al., (1999) J. Tissue Engineering 5(5):467-488) for use in promoting nerve growth. Examples of such materials include polyamide, methylcellulose, polyethyleneoxide block compolymers such as the Pluronics, especially F127 (BASF), collagen, and extracellular matrix (ECM) of the type sold as Biomatrix. Other useful materials include the polymer foams reported by Hadlock, et al., Laryngoscope (1999) 109(9):1412-1416.

Microfabrication techniques include micromachining, solid free form (SFF) techniques, and micromolding techniques, as well as other techniques based on well-established methods used to make integrated circuits, electronic packages and other microelectronic devices, having dimensions as small as a few nanometers and which can be mass produced at low per-unit costs.

Micromachining Techniques

Micromachining techniques are described in the literature, for example, by Rai-Choudhury, ed. Handbook of Microlithography, Micromaching & Microfabrication (SPIE Optical Engineering Press, Bellingham, Wash. 1997), the teachings of which are incorporated herein. The techniques can be used to form the device directly, or as discussed below, to form molds which are then used to form the devices.

Other microfabrication processes that may be used include lithography; etching techniques, such as wet chemical, dry, and photoresist removal; thermal oxidation; film deposition, such as evaporation (filament, electron beam, flash, and shadowing and step coverage), sputtering, chemical vapor deposition (CVD), epitaxy (vapor phase, liquid phase, and molecular beam), electroplating, screen printing, lamination, laser machining, and laser ablation (including projection ablation). See generally Jaeger, Introduction to Microelectronic Fabrication (Addison-Wesley Publishing Co., Reading Mass. 1988); Runyan, et al., Semiconductor Integrated Circuit Processing Technology (Addison-Wesley Publishing Co., Reading Mass. 1990); Proceedings of the IEEE Micro Electro Mechanical Systems Conference 1987-1998; Rai-Choudhury, ed., Handbook of Microlithography, Micromachining & Microfabrication (SPIE Optical Engineering Press, Bellingham, Wash. 1997).

Deep plasma etching can be used to create structures with diameters on the order of 0.1 μm or larger. In this process, an appropriate masking material is deposited onto a substrate and patterned into dots having the diameter of the desired tracts or channels. The wafer is then subjected to a carefully controlled plasma. Those regions protected by the metal mask remain and form the tracts.

Another method for forming devices including tracts or channels is to use microfabrication techniques such as photolithography, plasma etching, or laser ablation to make a mold form, transferring that mold form to other materials using standard mold transfer techniques, such as embossing or injection molding, and reproducing the shape of the original mold form using the newly-created mold to yield the final device. Alternatively, the creation of the mold form could be skipped and the mold could be microfabricated directly, which could then be used to create the final device.

Micromolding Techniques

Another method of fabricating tracts or channels utilizes micromold plating techniques. A photo-defined mold first is first produced, for example, by spin casting a thick layer, typically 150 μm, of an epoxy onto a substrate that has been coated with a thin sacrificial layer, typically about 10 to 50 nm. Arrays of cylindrical holes are then photolithographically defined through the epoxy layer, which typically is about 150 μm thick. (Despont, et al., “High-Aspect-Ratio, Ultrathick, Negative-Tone Near-UV Photoresist for MEMS,” Proc. of IEEE 10^{th} Annual International Workshop on MEMS, Nagoya, Japan, pp.518-522 (Jan. 26-30, 1997)). The diameter of these cylindrical holes defines the outer diameter of the tracts. The upper surface of the substrate, the sacrificial layer, is then partially removed at the bottom of the cylindrical holes in the photoresist. The exact method chosen depends on the choice of substrate. For example, the process has been successfully performed on silicon and glass substrates (in which the upper surface is etched using isotropic wet or dry etching techniques) and copper-clad printed wiring board substrates. In the latter case, the copper laminate is selectively removed using wet etching. Then a seed layer, such as Ti/Cu/Ti (e.g., 30 nm/200 nm/30 nm), is conformally DC sputter-deposited onto the upper surface of the epoxy mold and onto the sidewalls of the cylindrical holes. The seed layer should be electrically isolated from the substrate. Subsequently, one or more electroplatable metals or alloys, such as Ni, NiFe, Au, Cu, or Ti, are electroplated onto the seed layer. The surrounding epoxy is then removed, leaving molds which each have an interior annular hole that extends through the base metal supporting the tracts. The rate and duration of electroplating is controlled in order to define the wall thickness and inner diameter of the tracts.

The molds made as described above and injection molding techniques can be applied to form the tracts or channels in the molds (Weber, et al., “Micromolding—a powerful tool for the large scale production of precise microstructures”, Proc. SPIE—International Soc. Optical Engineer. 2879, 156-167 (1996); Schift, et al., “Fabrication of replicated high precision insert elements for micro-optical bench arrangements” Proc. SPIE—International Soc. Optical Engineer. 3513, 122-134 (1998). These micromolding techniques can provide relatively less expensive replication, i.e. lower cost of mass production.

Solid Free Form Manufacturing Techniques

As defined herein, SFF refers to any manufacturing technique that builds a complex three dimensional object as a series of two dimensional layers. The SFF methods can be adapted for use with a variety of polymeric, inorganic, and composite materials to create structures with defined compositions, strengths, and densities, using computer aided design (CAD).

Examples of SFF methods include stereo-lithography (SLA), selective laser sintering (SLS), ballistic particle manufacturing (BPM), fusion deposition modeling (FDM), and three dimensional printing (3DP). In a preferred embodiment, 3DP is used to precisely create channels and pores within a matrix to control subsequent cell growth and proliferation in the matrix of one or more cell types having a defined function, such as nerve cells.

The macrostructure and porous parameters can be manipulated by controlling printing parameters, the type of polymer and particle size, as well as the solvent and/or binder. Porosity of the matrix walls, as well as the matrix per se, can be manipulated using SFF methods, especially 3DP. Structural elements that maintain the integrity of the devices during erosion can also be incorporated. For example, to provide support, the walls of the device can be filled with resorbable inorganic material, which can further provide a source of mineral for the regenerating tissue. Most importantly, these features can be designed and tailored using computer assisted design (CAD) for individual patients to individualize the fit of the device.

Three Dimensional Printing (3DP).

3DP is described by Sachs, et al., “CAD-Casting: Direct Fabrication of Ceramic Shells and Cores by Three Dimensional Printing” Manufacturing Review 5(2), 117-126 (1992) and U.S. Pat. No. 5,204,055 to Sachs, et al., the teachings of which are incorporated herein. Suitable devices include both those with a continuous jet stream print head and a drop-on-demand stream print head. A high speed printer of the continuous type, for example, is the Dijit printer made and sold by Diconix, Inc., of Dayton, Ohio, which has a line printing bar containing approximately 1,500 jets which can deliver up to 60 million droplets per second in a continuous fashion and can print at speeds up to 900 feet per minute. Both raster and vector apparatuses can be used. A raster apparatus is where the printhead goes back and forth across the bed with the jet turning on and off. This can have problems when the material is likely to clog the jet upon settling. A vector apparatus is similar to an x-y printer. Although potentially slower, the vector printer may yield a more uniform finish. 3DP is used to create a solid object by ink jet printing a binder into selected areas of sequentially deposited layers of powder. Each layer is created by spreading a thin layer of powder over the surface of a powder bed. The powder bed is supported by a piston which descends upon powder spreading and printing of each layer (or, conversely, the ink jets and spreader are raised after printing of each layer and the bed remains stationary). Instructions for each layer are derived directly from a computer-aided design (CAD) representation of the component. The area to be printed is obtained by computing the area of intersection between the desired plane and the CAD representation of the object. The individual sliced segments or layers are joined to form the three dimensional structure. The unbound powder supports temporarily unconnected portions of the component as the structure is built but is removed after completion of printing.

As shown in U.S. Pat. No. 5,204,055, the 3DP apparatus includes a powder dispersion head which is driven reciprocally in a shuttle motion along the length of the powder bed. A linear stepping motor assembly is used to move the powder distribution head and the binder deposition head. The powdered material is dispensed in a confined region as the dispensing head is moved in discrete steps along the mold length to form a relatively loose layer having a typical thickness of about 100 to 200 microns, for example. An ink-jet print head having a plurality of ink-jet dispensers is also driven by the stepping motor assembly in the same reciprocal manner so as to follow the motion of the powder head and to selectively produce jets of a liquid binder material at selected regions which represent the walls of each cavity, thereby causing the powdered material at such regions to become bonded. The binder jets are dispensed along a line of the printhead which is moved in substantially the same manner as the dispensing head. Typical binder droplet sizes are between about 15 to 50 microns in diameter. The powder/binder layer forming process is repeated so as to build up the device layer by layer. While the layers become hardened or at least partially hardened as each of the layers is laid down, once the desired final part configuration is achieved and the layering process is complete, in some applications it may be desirable that the form and its contents be heated or cured at a suitably selected temperature to further promote binding of the powder particles. In either case, whether or not further curing is required, the loose, unbonded powder particles are removed using a suitable technique, such as ultrasonic cleaning, to leave a finished device. Finer feature size is also achieved by printing polymer solutions rather than pure solvents.

Stereo-lithography (SLA) and Selective Laser Sintering (SLS).

SFF methods are particularly useful for their ability to control composition and microstructure on a small scale for the construction of these medical devices. The SFF methods, in addition to 3DP, that can be utilized to some degree as described herein are stereo-lithography (SLA), selective laser sintering (SLS), ballistic particle manufacturing (BPM), and fusion deposition modeling (FDM).

Stereolithography is based on the use of a focused ultra-violet (UV) laser which is vector scanned over the top of a bath of a photopolymerizable liquid polymer material. The UV laser causes the bath to polymerize where the laser beam strikes the surface of the bath, resulting in the creation of a first solid plastic layer at and just below the surface. The solid layer is then lowered into the bath and the laser generated polymerization process is repeated for the generation of the next layer, and so on, until a plurality of superimposed layers forming the desired device is obtained. The most recently created layer in each case is always lowered to a position for the creation of the next layer slightly below the surface of the liquid bath. A system for stereolithography is made and sold by 3Systems, Inc., of Valencia, Calif., which is readily adaptable for use with biocompatible polymeric materials.

SLS also uses a focused laser beam, but to sinter areas of a loosely compacted plastic powder, the powder being applied layer by layer. In this method, a thin layer of powder is spread evenly onto a flat surface with a roller mechanism. The powder is then raster-scanned with a high-power laser beam. The powder material that is struck by the laser beam is fused, while the other areas of powder remain dissociated. Successive layers of powder are deposited and raster-scanned, one on top of another, until an entire part is complete. Each layer is sintered deeply enough to bond it to the preceding layer. A suitable system adaptable for use in making medical devices is available from DTM Corporation of Austin, Tex.

Ballistic Particle Manufacturing (BPM) and Fusion Deposition Modeling (FDM)

BPM uses an ink-jet printing apparatus wherein an ink-jet stream of liquid polymer or polymer composite material is used to create three-dimensional objects under computer control, similar to the way an ink-jet printer produces two-dimensional graphic printing. The device is formed by printing successive cross-sections, one layer after another, to a target using a cold welding or rapid solidification technique, which causes bonding between the particles and the successive layers. This approach as applied to metal or metal composites has been proposed by Automated Dynamic Corporation of Troy, N.Y.

FDM employs an x-y plotter with a z motion to position an extrudable filament formed of a polymeric material, rendered fluid by heat or the presence of a solvent. A suitable system is available from Stratasys, Incorporated of Minneapolis, Minn.

Polymer Materials, Binders and Solvents for Use in SSF Techniques

Depending on the processing method, the material forming the matrix may be in solution, as in the case of SLA, or in particle form, as in the case of SLS, BPM, FDM, and 3DP. In the preferred embodiment, the material is a polymer. In SLS, the polymer must be photopolymerizable. In the other methods, the material is preferably in particulate form and is solidified by application of heat, solvent, or binder (adhesive). In the case of SLS and FDM, it is preferable to select polymers having relatively low melting points, to avoid exposing incorporated bioactive agent to elevated temperatures.

A number of materials are commonly used to form a matrix. Unless otherwise specified, the term “polymer” will be used to include any of the materials used to form the matrix, including polymers and monomers which can be polymerized or adhered to form an integral unit, as well as inorganic and organic materials, as discussed below. In a preferred embodiment the particles are formed of a polymer which can be dissolved in an organic solvent and solidified by removal of the solvent, such as a synthetic thermoplastic polymer, for example, ethylene vinyl acetate, poly(anhydrides), polyorthoesters, polymers of lactic acid and glycolic acid and other α hydroxy acids, polyhydroxyalkanoates, and polyphosphazenes, a protein polymer, for example, albumin or collagen, or a polysaccharide. The polymer can be non-biodegradable or biodegradable, typically via hydrolysis or enzymatic cleavage. Examples of non-polymeric materials which can be used to form a part of the device or matrix for drug delivery include organic and inorganic materials such as hydoxyapatite, calcium carbonate, buffering agents, and lactose, as well as other common excipients used in drugs, which are solidified by application of adhesive or binder rather than solvent. In the case of polymers for use in making devices for cell attachment and growth, polymers are selected based on the ability of the polymer to elicit the appropriate biological response from cells, for example, attachment, migration, proliferation and gene expression.

Photopolymerizable, biocompatible water-soluble polymers include polyethylene glycol tetraacrylate (Mw 18,500) which can be photopolymerized with an argon laser under biologically compatible conditions using an initiator such as triethanolamine, N-vinylpyrrolidone, and eosin Y. Similar photopolymerizable macromers having a poly(ethylene glycol) central block, extended with hydrolyzable oligomers such as oligo(d,l-lactic acid) or oligo(glycolic acid) and terminated with acrylate groups, may be used.

Examples of biocompatible polymers with low melting temperatures include polyethyleneglycol 400 (PEG) which melts at 4-8° C., PEG 600 which melts at 20-25° C., and PEG 1500 which melts at 44-48° C. Another low melting material is stearic acid, which melts at 70° C.

Other suitable polymers can be obtained by reference to The Polymer Handbook, 3rd edition (Wiley, N.Y., 1989), the teachings of which are incorporated herein.

A preferred material is a polyester in the polylactide/polyglycolide family. These polymers have received a great deal of attention in the drug delivery and tissue regeneration areas for a number of reasons. They have been in use for over 20 years in surgical sutures, are Food and Drug Administration (FDA)-approved and have a long and favorable clinical record. A wide range of physical properties and degradation times can be achieved by varying the monomer ratios in lactide/glycolide copolymers: poly-L-lactic acid (PLLA) and poly-glycolic acid (PGA) exhibit a high degree of crystallinity and degrade relatively slowly, while copolymers of PLLA and PGA, PLGAs, are amorphous and rapidly degraded.

Solvents and/or binder are used in the preferred method, 3DP, as well as SLA and BPM. The binder can be a solvent for the polymer and/or bioactive agent or an adhesive which binds the polymer particles. Solvents for most of the thermoplastic polymers are known, for example, methylene chloride or other organic solvents. Organic and aqueous solvents for the protein and polysaccharide polymers are also known, although an aqueous solution, for example, containing a crosslinking agent such as carbodiimide or glutaraldehyde, is preferred if denaturation of the protein is to be avoided. In some cases, however, binding is best achieved by denaturation of the protein.

The binder can be the same material as is used in conventional powder processing methods or may be designed to ultimately yield the same binder through chemical or physical changes that take place in the powder bed after printing, for example, as a result of heating, photopolymerization, or catalysis.

These methods and materials are further described in PCTIUS96/09344 “Vascularized TissueRegeneration Matrices Formed by Solid Free-Form Fabrication Methods” Massachusetts Institute of Technology and Children's Medical Center Corporation.

2. Fibrous Scaffolds for Implantation

Fibrous scaffolding can be used to implant the cells, for example, as described in U.S. Pat. No. 5,759,830 to Vacanti, et al. The design and construction of the scaffolding is of primary importance. The matrix should be a pliable, non-toxic, porous template for vascular ingrowth. The pores should allow vascular ingrowth and the injection of cells into the scaffold without damage to the cells or patient. The scaffolds are generally characterized by interstitial spacing or interconnected pores in the range of at least between approximately 100 and 300 microns in diameter. The matrix should be shaped to maximize surface area, to allow adequate diffusion of nutrients and growth factors to the cells and to allow the ingrowth of new blood vessels and connective tissue.

The same type of polymers can be used as in the Solid Free Form Manufacturing techniques described above. In the preferred embodiment, the matrix is formed of a bioabsorbable, or biodegradable, synthetic polymer such as a polyanhydride, polyorthoester, polyhydroxy acid such as polylactic acid, polyglycolic acid, or a natural polymer like polyalkanoates such as polyhydroxybutyrate and copolymers or blends thereof. Proteins such as collagen can be used, but is not as controllable and is not preferred. These materials are all commercially available. Non-biodegradable polymers, including polymethacrylate and silicon polymers, can be used, depending on the ultimate disposition of the growing cells.

In some embodiments, attachment of the cells to the polymer is enhanced by coating the polymers with compounds such as basement membrane components, agar, agarose, gelatin, gum arabic, collagens types I, II, III, IV, and V, fibronectin, laminin, glycosaminoglycans, mixtures thereof, and other materials, especially attachment peptides and polymers having attachment peptides or other cell surface ligands bound thereto, known to those skilled in the art of cell culture. Vitrogen-100 collagen (PCO 701) has been used in these experiments.

3. Hydrogel Matrices for Implantation Polymeric materials which are capable of forming a hydrogel can be utilized. The polymer is mixed with cells for implantation into the body and is permitted to crosslink to form a hydrogel matrix containing the cells either before or after implantation in the body. In one embodiment, the polymer forms a hydrogel within the body upon contact with a crosslinking agent. A hydrogel is defined as a substance formed when an organic polymer (natural or synthetic) is crosslinked via covalent, ionic, or hydrogen bonds to create a three-dimensional open-lattice structure which entraps water molecules to form a gel. Naturally occurring and synthetic hydrogel forming polymers, polymer mixtures and copolymers may be utilized as hydrogel precursors. See for example, U.S. Pat. No. 5,709,854 and WO 94/25080 by Reprogenesis.

In one embodiment, calcium alginate and certain other polymers that can form ionic hydrogels which are malleable. For example, a hydrogel can be produced by cross-linking the anionic salt of alginic acid, a carbohydrate polymer isolated from seaweed, with calcium cations, whose strength increases with either increasing concentrations of calcium ions or alginate. The alginate solution is mixed with the cells to be implanted to form an alginate suspension which is injected directly into a patient prior to hardening of the suspension. The suspension then hardens over a short period of time due to the presence in vivo of physiological concentrations of calcium ions. Modified alginate derivatives, for example, more rapidly degradable or which are derivatized with hydrophobic, water-labile chains, e.g., oligomers of ε-caprolactone, may be synthesized which have an improved ability to form hydrogels. Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Additional examples of materials which can be used to form a hydrogel include polyphosphazines and polyacrylates, which are crosslinked ionically, or block copolymers such as Pluronics™ or Tetronics™, polyethylene oxide-polypropylene glycol block copolymers which are crosslinked by temperature or pH, respectively. Other materials include proteins such as fibrin, polymers such as polyvinylpyrrolidone, hyaluronic acid and collagen. Polymers such as polysaccharides that are very viscous liquids or are thixotropic, and form a gel over time by the slow evolution of structure, are also useful. For example, hyaluronic acid, which forms an injectable gel with a consistency like a hair gel, may be utilized. Modified hyaluronic acid derivatives are particularly useful. Polymer mixtures also may be utilized. For example, a mixture of polyethylene oxide and polyacrylic acid which gels by hydrogen bonding upon mixing may be utilized. In one embodiment, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

Covalently crosslinkable hydrogel precursors also are useful. For example, a water soluble polyamine, such as chitosan, can be cross-linked with a water soluble diisothiocyanate, such as polyethylene glycol diisothiocyanate. The isothiocyanates will react with the amines to form a chemically crosslinked gel. Aldehyde reactions with amines, e.g., with polyethylene glycol dialdehyde also may be utilized. A hydroxylated water soluble polymer also may be utilized.

Alternatively, polymers may be utilized which include substituents which are crosslinked by a radical reaction upon contact with a radical initiator. For example, polymers including ethylenically unsaturated groups which can be photochemically crosslinked may be utilized, as disclosed in WO 93/17669. Additionally, water soluble polymers which include cinnamoyl groups which may be photochemically crosslinked may be utilized, as disclosed in Matsuda et al, ASAID Trans., 38:154-157 (1992).

II. Methods for Engineering and Implantation of Cells

A. Disorders to be Treated

A variety of conditions that produce abnormal growth, including treatment of malignant and benign neoplasias, vascular malformations (hemangiomas), inflammatory conditions including those resulting from infection, especially chronic or recalcitrant conditions such as those in the sinuses or which are cystic, keloid formation, endometriosis, congenital or endocrine abnormalities such as testotoxicosis (Teixeira et al, PNAS 1999) and other conditions that produce abnormal growth, can be treated. In a preferred embodiment, the disorder is characterized by pre-cancerous, cancerous or neoplastic cells, or the presence of a tumour. The disorder can affect an epithelial tissue, e.g., skin, e.g., the dermis or epidermis. In other preferred embodiments, the disorder affects the breast, prostate, lung, stomach or bowel.

In one preferred embodiment, the disorder is a cancerous cell growth, e.g., a squamous cell carcinoma of the skin, malignant melanoma, prostate cancer, breast cancer, colon cancer, lung cancer (e.g., non-small cell lung cancer), or Kaposi's sarcoma. In a particularly preferred example, the disorder is characterized by unwanted skin cell proliferation, e.g., cancer of the skin, e.g., a squamous cell carcinoma of the skin, or a malignant melanoma. In another preferred embodiment, the disorder is characterized by unwanted prostate cell proliferation, e.g., cancer of the prostate.

In another preferred embodiment, the disorder is characterized by benign unwanted cell proliferation, e.g., unwanted skin proliferation in the skin, e.g., psoriasis or papilloma formation. The method can include increasing TSP-2 activity, thereby inhibiting unwanted proliferation, e.g., unwanted proliferation in the skin. In another embodiment, the disorder is an inflammatory disorder associated with angiogenesis. For example, the disorder can be psoriasis, rheumatoid arthritis or multiple sclerosis. The method can include increasing TSP-2 activity, thereby treating the inflammatory disorder. In still another embodiment, the disorder is characterized by unwanted angiogenesis, e.g., unwanted angiogenesis of the eye. For example, the disorder can be a retinal disorder characterized by unwanted angiogenesis such as diabetic retinopathy. In other embodiments, the disorder can be, for example, restenosis after coronary angioplasty.

Coupled with minimally invasive delivery systems, the biodegradable implants producing the therapeutic proteins from transfected autologous cells can be introduced into a variety of sites to deliver therapeutics, particularly where a local effect is advantageous. This allows use of a variety of recombinant proteins without the need for complex purification protocols. The method also allows cells to be engineered to increase expresson/produce one molecules, such as TSP-2, while inhibiting production or expression of another molecule. For example, in a preferred embodiment, the method of increasing TSP-2 production further includes inhibiting VEGF activity. VEGF activity can be decreased, e.g., by administering: a VEGF nucleic acid molecule, e.g., an antisense molecule or VEGF ribozyme, that can bind to cellular VEGF mRNA and inhibit expression of the protein, e.g., by inhibiting transcription of VEGF; an antibody that specifically binds to VEGF protein, e.g., an antibody that disrupts VEGF's ability to bind to its natural cellular target; a dominant negative VEGF protein or fragment thereof; or an agent which decreases VEGF nucleic acid expression, e.g., a small molecule which binds the promoter of VEGF.

B. Engineering of Cells

In the preferred embodiment, patient cells are transfected with the gene to be expressed, for example, rTSP-2 cDNA, to produce cells having stably incorporated therein the DNA encoding the molecules to be expressed. Methods yielding transient expression, such as most adenoviral vectors, are not preferred. Stable transfectants are obtained by culturing and selection for expression of the encoded molecule(s). Those cells that exhibit stable expression are seeded onto/into the appropriate matrix and then implanted using techniques such as those described in the following examples.

C. Seeding of Matrices

The level of expression of the bioactive molecules is measured prior to implantation to insure that an adequate number of cells is implanted. In general, the higher the number of cells implanted, the better. Cells are preferably cultured initially in vitro, then implanted before the matrix degrades but when the level of bioactive molecules is highest. An example of a suitable seeding density is between 1 and 10×10⁶ cells on a matrix with a surface area of 0.25 cm².

D. Implantation of Matrices

The devices are implanted into the patient at the site in need of treatment using standard surgical techniques. In one embodiment, the device is constructed, seeded with cells, and cultured in vitro prior to implantation. The cells are cultured in the device, tested for high production of the anti-angiogenic molecules by a standard technique such as ELISA, then implanted.

The technique described herein can be used for delivery of many different cell types for different purposes. The matrix may be implanted in one or more different areas of the body to suit a particular application. Matrices with hepatocytes or other high oxygen organ cells may be implanted into the mesentery to insure a good blood supply. Sites other than the mesentery for injection or implantation of cells include the ovarian pedicle, subcutaneous tissue, retroperitoneum, properitoneal space, and intramuscular space.

The need for these additional procedures depends on the particular clinical situation.

### III. EXAMPLES

The present invention will be further understood by reference to the following non-limiting examples.

### Example 1

### Engineering of Cells to Produce an effective amount of TSP-2 to Inhibit Tumor Growth following Implantation on and within Biodegradable Polymeric Matrices.

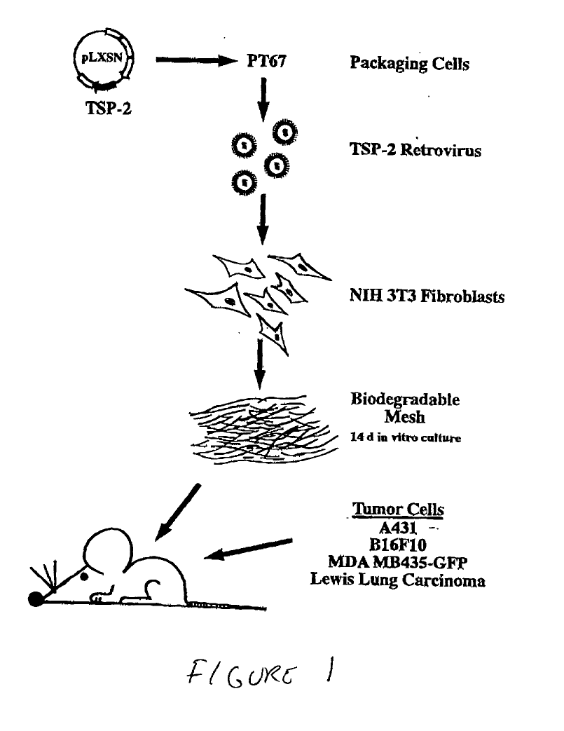

To achieve efficient inhibition of malignant tumor growth via continuously elevated systemic TSP-2 levels, a cell-based, anti-angiogenic, tissue-engineered gene therapy approach for the in vivo production of full-length TSP-2 within the living tumor host was developed. The process is shown schematically in FIG. 1. Tissue engineered implants, which contained large numbers of TSP-2 transduced fibroblasts grown on biodegradable polymer scaffolds, as described by Vacanti, J.P. et al. Pediatr. Surg. 23, 3 (1988), efficiently secreted TSP-2 into the blood circulation after intraperitoneal implantation. Bioimplants maintained TSP-2 secretion over prolonged time periods, resulting in a potent inhibition of tumor growth and angiogenesis of three different, highly aggressive malignant tumors implanted at a distant site.

Experimental protocol

Cell culture.

NIH 3T3 fibroblasts, the human squamous cell carcinoma cell line A431, and the murine melanoma cell line B16F10 were obtained from the American Type Culture Collection (Rockville, Md.). The murine Lewis lung carcinoma cell line (LLC) was kindly provided by Dr. J. Lawler (Beth Israel Deaconess Medical Center, Boston, Mass.). The packaging cell line RetroPack™ PT67 was purchased from Clontech Laboratories Inc. (Palo Alto, Calif.). NIH 3T3, A431, B16F10 and LLC were maintained in Dulbecco's modified Eagle's medium (DMEM) containing 10% fetal bovine serum, 4.5 mg/ml glucose, 2 mM L-glutamine, 100 U/ml penicillin G, and 100 μg/ml streptomycin (Life Science, Grand Island, N.Y.). PT67 cells were cultivated in the same medium with addition of 100 mM sodium pyruvate (Life Science).

Cell transfection.

The retroviral transfection plasmid containing the full-length murine TSP-2 cDNA was prepared by ligation of a 3.6-kbp EcoRI fragment Streit, M., et al., into the EcoRI site of a pLXSN retroviral transfection vector (Clontech) which contains a neomycin selection cassette. PT67 packaging cells were transfected with the TSP-2/pLXSN retroviral plasmid or with unaltered pLXSN, using the SuperFect™ transfection reagent (Qiagen, Chatsworth, Calif.) and 10 μg of each plasmid according to the manufacturer's recommendations. The cells were selected in medium containing 800 μg/ml G418 (Sigma, St. Louis, Mo.) for 3-4 weeks, and more than 60 resistant clones were isolated. Retroviral titers were determined by incubation of NIH 3T3 cells with 10-fold serial dilutions of culture supernatants obtained from TSP-2/pLXSN and pLXSN control transfected clones. PT67 cell clones with a viral titer of greater than 10⁶ cfu/ml were used for further experiments. For retroviral transfection, NIH 3T3 cells were incubated with virus-containing supernatants in the presence of 8 μg/ml polybrene (Sigma) for 6 hours on 4 consecutive days and were then subjected to G418 selection.

Preparation of polymer-cell grafts.

Biodegradable polymer grafts were prepared from 1 mm thick sheets of nonwoven fibers of polyglycolic acid (density 70 mg/cm³, fiber diameter 14 μm, average pore size 250 μm, Smith and Nephew, York County, UK). Sheets were sectioned into 0.5 cm² squares which were placed into 12-well tissue culture plates (Costar, Cambridge, Mass.), sterilized with 95% ethanol, and washed with phosphate-buffered saline. Sterile 1 sodium hydroxide was added to each well for 60 seconds to render the polymer hydrophilic. Thereafter, the polymer was washed with distilled water and was coated with 30 μg/ml collagen type I (Vitrogen™, Collagen Biomaterials, Palo Alto, Calif.) for 1 h. Transfected NIH 3T3 fibroblasts were trypsinized, resuspended in culture medium and 1.5×10⁷ cells were seeded onto each polymer square. After 2 hours at 37° C. and 5% CO₂, fresh culture medium was added. Polymer-cell grafts were cultivated in vitro for 14 days before grafting.

Northern Blot Analysis.

Total cellular RNA was isolated from confluent NIH 3T3 cell cultures using the RNeasy kit™ (Qiagen). Ten μg of RNA were fractionated by electrophoresis on 1% agarose formaldehyde gels and were transferred to Biotrans™ nylon supported membranes (ICN Pharmaceuticals, Costa Mesa, Calif.) as described in Streit, M., et al., ³²P-radiolabeled cDNA probes were prepared with a random primed synthesis kit (Multiprime™; Amersham, Arlington Heights, Ill.) using a 4.19-kbp mouse TSP-2 cDNA and a 950-bp mouse VEGF cDNA as templates. A 2.0-kbp human β-actin cDNA probe (Clontech) was used as a control for equal RNA loading. Blots were washed at high stringency and were exposed to X-OMAT MR™ film (Kodak, Rochester, N.Y.) for varying times.

Implantation of Polymer-cell Grafts and Tumorigenesis Assay.

Fourteen days after seeding with TSP-2 or control fibroblasts, 5×5>1 mm polymer squares were implanted into the right ovarian pedicle of 8-week-old female BALB/c (nu/nu) nude mice as described in Kristjansen, P. E., et al., Microvasc. Res. 48, 389-402 (1994). After induction of anesthesia with a mixture of ketamine (800 μg/10 g body weight, Ketaset™, Fort Dodge Laboratories, Fort Dodge, Iowa) and avertin (0.5 μg/10 g body weight 2,2,2-tribromethanol in 2.5% t-amyl alcohol, Sigma), a one centimeter horizontal incision was made in the right flank and the ovarian pedicle was identified and delivered out of the wound. The polymer-cell graft was laid on the ovarian pedicle and sutured on place with 6-0 prolene suture (Roboz Surgical, Rockville, Md.). The abdomen was closed with 9 mm autoclips (Becton Dickinson, Sparks, Md.). One week after implantation of cell-seeded bioimplants, A431 squamous cell carcinoma cells (2×10⁶) or B16F10 malignant melanoma cells (2×10⁶) were injected intradermally and Lewis lung carcinoma cells (0.5×10⁶) were injected subcutaneously into both flanks of nude mice (2 sites per mouse; five mice per cell line and per type of bioimplant). The smallest and largest tumor diameter were measured weekly, using a digital caliper, and tumor volumes were calculated using the following formula: Volume=4/3×π×(½×smaller diameter)²×½×larger diameter. Tumor data were analyzed by the two-sided unpaired t-test. Mice were sacrificed after 3 to 4 weeks. All animal studies were approved by the Massachusetts General Hospital Subcommittee on Research Animal Care.

Western Blot Analysis.

Conditioned media were obtained from confluent fibroblast cultures grown for 48 hours in serum-free DMEM. Culture supernatants were also collected from fibroblast-populated polymer scaffolds every 48 hours. Serum samples were obtained from mice bearing bioimplants with either TSP-2 overexpressing or control fibroblasts 4 and 5 weeks after implantation. TSP-2 was concentrated as described by Streit, M., et al. Proc. Natl. Acad. Sci. USA 96, 14888-14893 (1999), and samples were boiled in denaturing sample buffer. Fifteen μl of each sample were electrophoresed on polyacrylamide gels and were blotted onto polyvinylidene difluoride membranes (Bio-Rad). To verify equal protein loading, membranes were stained with 0.1% Ponceau red (Sigma) diluted in 5% acetic acid. Membranes were incubated overnight in PBS containing 0.1% Tween-20 and 3% bovine serum albumin to block nonspecific binding. Membranes were then incubated with anti-TSP-2 antibody (clone N-20, Santa-Cruz Biotechnology, Santa Cruz, Calif.), washed in PBS/Tween, incubated with horseradish peroxidase-conjugated anti-goat IgG (Santa Cruz), and were analyzed by the enhanced-chemiluminescence system (Amersham).

In situ Hybridization and Immunohistochemistry.

In situ hybridization was performed on 6 μm cryosections of fibroblast-seeded bioimplants 5 weeks after implantation as described in Streit, M., et al. The sense and antisense single-stranded RNA-probes for mouse TSP-2 were transcribed from a pBluescript™ II KS+ vector containing a 290-bp fragment of the coding region of mouse TSP-2. Immunohistochemical and immunofluorescence stainings were performed on 6 μm frozen sections of tumors and polymer-cell grafts as described by Streit, et al., using a polyclonal anti-TSP-2 antibody (Santa Cruz), a polyclonal rabbit anti-mouse TSP-2 antibody (kindly provided by Dr. Paul Bornstein, University of Washington, Seattle), or a rat monoclonal antibody against mouse CD31 (Phiarmingen).

Computer-assisted Morphometric Analysis of Blood Vessels.

Representative sections were obtained from five TSP-2- or controltreated tumors for each tumor cell line and from five control or TSP-2 overexpressing bioimplants. Sections were stained with an anti-mouse CD31 monoclonal antibody and were analyzed using a Nikon E-600 microscope (Nikon, Melville, N.Y.). Images were captured with a Spot digital camera (Diagnostic Instruments, Sterling Heights, Mich.) and morphometric analyses were performed as described in Streit, M., et al., using the IP LAB software (Scanalytics, Billerica, Mass.). The two-sided unpaired t-test was used to analyze differences in microvessel density and vascular size and area.

Results

Retroviral Expression of TSP-2 in Fibroblasts.

To generate a retroviral transfection system for the overexpression of the angiogenesis inhibitor thrombospondin-2 (TSP-2), the packaging cell line PT67 was first transduced with a TSP-2-pLXSN expression vector. Supernatants obtained from five TSP-2-pLXSN transfected PT67 clones with high retroviral titers were used to stably transfect NIH 3T3 fibroblasts. Northern blot analysis revealed low constitutive expression of TSP-2 mRNA in fibroblasts transfected with empty pLXSN expression vector (control) whereas strong TSP-2 mRNA expression was detected in all TSP-2 transfected clones. Northern blot analysis confirmed TSP-2 mRNA overexpression in fibroblast clones stably transfected with a retroviral TSP-2 expression vector and only low levels of endogenous TSP-2 expression in vector control clones). Western Blot analysis of conditioned media confirmed selective TSP-2 secretion by fibroblast clones transfected with a retroviral TSP-2 expression vector. Western blot analysis of cell culture supernatants confirmed efficient TSP-2 secretion by TSP-2 transfected but not by vector control transfected fibroblasts at 10 to 14 days after grafting onto synthetic biodegradable polymer fibers in vitro. Overexpression of TSP-2 did not affect the mRNA expression of the angiogenesis factor VEGF. The TSP-2 transfected fibroblast clone 50 and the control clone 6 were seeded onto biodegradable polymer scaffolds and were cultured for 14 days before grafting. Culture supernatants were analysed for TSP-2 expression every 48 hours after seeding, and efficient TSP-2 secretion by TSP-2 transfected fibroblasts was detected after 10 to 14 days in culture. In contrast, little or no TSP-2 was secreted by control transfected fibroblasts after seeding onto the polymer.

In vivo Growth of Transfected Fibroblasts on Biodegradable Polymer Grafts.

After 14 days of in vitro culture, polymer-cell grafts of transfected fibroblasts were implanted into the ovarian pedicle in the peritoneal cavity of nude mice. The ovarian pedicle was chosen as an easily accessible and well-vascularized tissue bed for implantation. The transplanted polymer-cell grafts formed well-vascularized, encapsuled fibroblast nodules. Polymer-cell grafts were implanted into the peritoneal cavity of nude mice and remained in situ for 5 weeks. Implanted polymer-grafts formed well vascularized and encapsuled nodules. Cross-sections of fibroblast nodules revealed no signs of tissue necrosis. Histological analysis revealed a comparable histological differentiation and architecture with densely packed fibroblasts in both control and TSP-2 overexpressing polymer-cell grafts (Hematoxylin/eosin staining). Immunostaining with an anti-CD31 monoclonal antibody demonstrated efficient ingrowth of blood vessels in polymer-cell grafts derived from control transfected and from TSP-2 overexpressing fibroblasts. Cross-sections of fibroblast nodules revealed no sign of tissue necrosis. No size differences were detected between TSP-2 overexpressing and control bioimplants. Histological analysis revealed a comparable histo-architecture and density of fibroblasts in TSP-2 transfected and control bioimplants. No remnants of biodegradable polymer fibers were detected in polymer-cell grafts 5 weeks after implantation, indicating complete dissolution of the temporary cell delivery scaffolds. Immunostaining for the endothelial junction molecule CD31 revealed efficient ingrowth of blood vessels into all polymer-cell grafts examined.

Expression and Secretion of TSP-2 by Polymer-cell Grafts In Vivo.

In situ hybridization analyses and immunofluorescence stainings confirmed that TSP-2 transfected fibroblasts maintained TSP-2 mRNA and protein expression for at least 5 weeks in vivo. Bioimplants of TSP-2 transfected fibroblasts showed high levels of TSP-2 mRNA expression whereas only weak TSP-2 mRNA expression was detected in control polymer-cell grafts. TSP-2 mRNA and protein expression were maintained in TSP-2 transfected fibroblast grafts after 5 weeks of in vivo growth. In situ hybridization confirmed strong TSP-2 mRNA expression in polymer cell-grafts derived from TSP-2 fibroblasts whereas only weak TSP-2 mRNA expression was detected in control grafts. Immunofluorescence double-staining for CD31 and TSP-2 confirmed strong TSP-2 protein expression in TSP-2 transfected but not in control transfected fibroblast grafts, without any major differences in the number and size of CD31-stained blood vessels. In accordance with these findings, strong cellular TSP-2 immunoreactivity was found in TSP-2 overexpressing implants throughout the whole graft but little or no TSP-2 protein expression was detected in control grafts. Levels of serum TSP-2 were evaluated 4 and 5 weeks after implantation of the polymer-cell grafts. Western blot analysis revealed high levels of circulating TSP-2 in the serum of mice bearing TSP-2 overexpressing bioimplants whereas no TSP-2 was detected in the serum of mice transplanted with control polymer-cell grafts. Specifically, representative Western blot analysis detected circulating TSP-2 protein only in serum samples obtained from mice bearing TSP-2 transfected bioimplants. mmunofluorescence double-staining for CD31 and TSP-2 confirmed only sparse TSP-2 expression in the mesenchymal stroma of A431 squamous cell carcinomas grown in mice bearing control bioimplants. Accumulation of TSP-2 protein was found within and surrounding blood vessels in mice bearing TSP-2 secreting bioimplants.

Inhibition of Tumor Growth in Mice Bearing TSP-2 Secreting Bioimplants.

Seven days after implantation of the polymer-cell grafts, A431 squamous cell carcinoma cells, B16F10 malignant melanoma cells and Lewis lung carcinoma cells were injected into both flanks of the graft-bearing mice. In mice bearing control grafts, A431 and B16F10 cells formed rapidly growing malignant tumors which reached a volume of 1,500 mm³ -2,500 mm³ within 3 weeks. In contrast, tumor growth was significantly inhibited (P<0.001) by more than 60% in mice bearing TSP-2 secreting bioimplants. A more than 70% inhibition of malignant tumor growth by cell-based TSP-2 gene-therapy was observed after implantation of Lewis lung carcinoma cells. Differential immunofluorescence staining for TSP-2 and CD31 revealed only sparse TSP-2 expression in the mesenchymal stroma of A431 squamous cell carcinomas in mice bearing control bioimplants whereas strong TSP-2 protein expression was detected both in and surrounding tumor blood vessels of A431 carcinomas in mice bearing TSP-2 secreting bioimplants.

Inhibition of Tumor Angiogenesis in Mice Bearing TSP-2 Secreting Bioimplants.

The effect of cell-based TSP-2 gene-therapy on tumor angiogenesis was determined. Immunostainings for the endothelial junction molecule CD31 revealed a marked reduction of the size of tumor microvessels in mice bearing TSP-2 secreting bioimplants as compared to control mice. These effects on tumor vessel size were detected in A431 squamous cell carcinomas, in B16F10 malignant melanomas and in Lewis lung carcinomas. Inhibition of tumor angiogenesis by cell-based TSP-2 gene therapy. Immunostaining with an anti-CD31 antibody demonstrated rarefication of tumor blood vessels in mice bearing TSP-2 secreting bioimplants. Reduced vascularization was found in A431 squamous cell carcinomas, B16F10 malignant melanomas and Lewis lung carcinomas. Computer-assisted image analysis of representative digital images revealed that the average vessel size and the relative tumor area occupied by blood vessels (total vascular area) were significantly reduced (p<0.01) by circulating TSP-2 in A431 squamous cell carcinomas, in B16F10 malignant melanomas, and in Lewis lung carcinomas. The average vessel density was slightly reduced after continuous TSP-2 treatment.

Discussion

An anti-angiogenic, cell-based gene therapeutical approach has been established. Biodegradable polymer implants serve as temporary scaffolds, capable of supporting the growth of large numbers of genetically modified fibroblasts to transform normal tissue into a “factory” for the continuous in vivo secretion of the anti-angiogenic agent TSP-2. These results show that fibroblasts can be retrovirally transduced to overexpress TSP-2 and continue to grow on synthetic biodegradable polymer fibers after intraperitoneal implantation. The transplanted bioimplants formed well vascularized nodules that maintained TSP-2 overexpression in vivo as determined by in situ hybridization and immunofluorescence analysis. Moreover, it was confirmed that increased systemic serum concentrations of TSP-2 reached biologically active levels as revealed by the significant growth inhibition of three different, highly aggressive tumor cell lines implanted at a distant site. These tumor cell lines were chosen because of their angiogenesis-dependent in vivo growth Streit, M., et al., Am. Pathol. 155, 441-452 (1999), and Boehm, T., et al., Nature 390, 404-407 (1997). TSP-2 expression and serum levels were sustained for at least five weeks and were sufficient to significantly inhibit tumor angiogenesis in all three tumor models, as measured by a significant reduction of tumor vascularization. Together with the observed accumulation of TSP-2 protein within and adjacent to angiogenic tumor vessels, these findings clearly indicate that bioimplant-derived TSP-2 inhibited tumor growth via inhibition of angiogenesis. It is of interest that circulating TSP-2 significantly inhibited neovascularization in malignant tumors whereas no effect was found on the angiogenesis within the fibroblast transplants. Histologically, the highly vascularized fibroblast transplants resembled the granulation tissue of healing wounds which also consists primarily of fibroblasts and vascular endothelial cells Martin, P., Science 276, 671-674 (1997). In accordance with these findings, it has been reported that treatment with endostatin, a potent tumor angiogenesis inhibitor, did not reduce the blood vessel density in wound granulation tissue, Berger, A. C. et al. J. Surg. Res. 91, 26-31 (2000), suggesting a different sensitivity of angiogenic blood vessels in malignant tumors and in wound granulation tissue to specific anti-angiogenic treatments.

Modifications and variations of the present invention are intended to come within the scope of the following claims.

We claim:

1. A method for treating a disorder characterized by excessive proliferation of tissue comprising
implanting a cell-matrix structure comprising a matrix having attached thereto an effective amount of cells stably expressing a gene encoding an anti-angiogenic molecule in an amount effective to inhibit or regress the excessive tissue proliferation,
wherein the cells are either genetically engineered to produce the anti-angiogenic molecule or of a different cell type than the tissue that has proliferated excessively and naturally produce the anti-angiogenic molecule.

2. The method of claim 1 wherein the disorder is selected from the group consisting of malignant and benign neoplasias, vascular, inflammatory conditions causing excessive proliferation of cells, keloid formation, intraperitoneal or intrathoracic adhesions, endometriosis, congenital or endocrine abnormalities, psoriasis, unwanted skin proliferation, rheumatoid arthritis, multiple sclerosis, unwanted angiogenesis of the eye, restenosis, and infections causing excessive proliferation of cells.

3. The method of claim 1 wherein the matrix is selected from the group consisting of fibrous scaffolds, polymeric hydrogels, and micromachine or micromolded substrates.

4. The method of claim 1 wherein the cells are selected from the group consisting of fibroblasts, tissue specific cells, progenitor cells, and stem cells.

5. The method of claim 4 wherein the cells are genetically engineered to produce the anti-angiogenic molecule.

6. The method of claim 5 wherein the anti-angiogenic molecule is thrombomodulin.

7. The method of claim 1 wherein the anti-angiogenic molecule is endogenous to the cells on the matrix and the cells are engineered to increase expression of the anti-angiogenic molecule.

8. A cell-matrix structure for implantation into a patient having attached thereto an effective amount of cells stably expressing a gene encoding an anti-angiogenic molecule in an effective amount to inhibit or regress excessive tissue proliferation in a patient in need thereof, wherein the cells are either genetically engineered to produce the anti-angiogenic molecule or of a different cell type than the tissue that has proliferated excessively which produces the anti-angiogenic molecule.

9. The cell-matrix structure of claim 8 wherein the cells produce a anti-angiogenic molecule effective to treat a disorder is selected from the group consisting of malignant and benign neoplasias, vascular, inflammatory conditions causing excessive proliferation of cells, keloid formation, intraperitoneal or intrathoracic adhesions, endometriosis, congenital or endocrine abnormalities, psoriasis, unwanted skin proliferation, rheumatoid arthritis, multiple sclerosis, unwanted angiogenesis of the eye, restenosis, and infections causing excessive proliferation of cells.

10. The cell-matrix structure of claim 8 wherein the matrix is selected from the group consisting of fibrous scaffolds, polymeric hydrogels, and micromachine or micromolded substrates.

11. The cell-matrix structure of claim 8 wherein the cells are selected from the group consisting of fibroblasts, tissue specific cells, progenitor cells, and stem cells.

12. The cell-matrix structure of claim 8 wherein the cells are genetically engineered to produce the anti-angiogenic molecule.

13. The cell-matrix structure of claim 8 wherein the anti-angiogenic molecule is thrombomodulin.

14. The cell-matrix structure of claim 8 wherein the anti-angiogenic molecule is endogenous to the cells on the matrix and the cells are engineered to increase expression of the anti-angiogenic molecule.

15. The cell-matrix structure of claim 8 wherein the cells are selected based on natural production of the wherein the anti-angiogenic molecule is endogenous to the cells on the matrix and the cells are engineered to increase expression of the anti-angiogenic molecule and the cells are implanted at a site where the wherein the anti-angiogenic molecule is endogenous to the cells on the matrix and the cells are engineered to increase expression of the anti-angiogenic molecule in an amount effective to inhibit proliferation or cause tissue regression.
